(19) United States
(12) Patent Application Publication
(10) Pub. No.: US 2023/0041020 A1
(43) Pub. Date: Feb. 9, 2023

(54) PROCESS FOR PRODUCING A NONWOVEN FROM BACTERIAL NANOCELLULOSE

(71) Applicant: Kim Köster & Cord Henning Labuhn GbR, Berlin (DE)

(72) Inventor: Cord LABUHN, Berlin (DE)

(73) Assignee: Kim Köster & Cord Henning Labuhn GbR, Berlin (DE)

(21) Appl. No.: 17/787,944

(22) PCT Filed: Dec. 23, 2020
(86) PCT No.: PCT/EP2020/087745

§ 371 Date: Jun. 22, 2022

(30) Foreign Application Priority Data
Dec. 23, 2019 (IB) PCT/IB2019/061260

(51) Int. Cl. C08J 3/075; C12P 19/04; C08L 1/02; D04H 1/425

(52) U.S. Cl. CPC C08J 3/075; C12P 19/04; C08L 1/02; D04H 1/425; C08J 2301/02; D10B 2509/00

(57) ABSTRACT

The invention relates to a process for the production of a dimensionally stable hydrogel consisting of bacterial nanocellulose, with the steps of providing a sugar-containing solution, inoculating said sugar-containing solution with a strain of bacteria, culturing said solution and washing the nonwoven material resulting from the culturing.

The invention relates to a process for the production of a hydrogel consisting of bacterial nanocellulose, with the steps of providing a sugar-containing solution, inoculating said sugar-containing solution with a strain of bacteria, culturing said solution and washing the hydrogel resulting from the culturing, and a nonwoven material produced using the aforementioned processes.

Dimensionally stable hydrogels made of bacterial nanocellulose are known. This bacterial nanocellulose is produced by culturing a suitable strain of bacteria in an aqueous and acidic buffered nutrient medium, with a dimensionally stable hydrogel forming at the interface between the nutrient medium and the air during the culturing, which can sometimes last for several weeks. Depending on the production process, hydrogels produced in this way also meet the requirements for vegan products. These dimensionally stable hydrogels are also referred to below as “nanocellulose gels”.

Due to their structural similarity to human skin, their good compatibility with the human organism and their high water retention capacity, nanocellulose gels can be used, among other things, as massage sponges, washcloths, wet wipes or protective films. Nanocellulose gels can be loaded during growth and equipped with special properties (such as color, taste, scent, surface structure, permeability, active substance loading). In addition, in-situ modification, i.e. influencing the synthesis during the ongoing culturing process by adding different additives to the nutrient medium, is known. It is also known to modify biomaterials based on bacterial nanocellulose following their synthesis (post-modification).

For this purpose, an instant powder was developed within the scope of the invention, which greatly simplifies the domestic production of nanocellulose gels. Dissolved in water, this powder is also suitable for subsequent loading and modification of the nanocellulose. In the presence of the instant solution, a correspondingly cultured strain of bacteria initiates the growth of a new probiotic ‘active’ nanocellulose gel and later serves for hygienic storage and renovation thereof.

‘Microbial polymers’ include polymers produced by a microorganism such as bacteria, fungi or algae. Nanocellulose is preferably synthesized by culturing microbial strains such as Gluconacetobacter, Enterobacter, Agrobacterium, Pseudomonas and Rhizpobium. In addition to Gluctonacetobacter hansenii and Gluconacetobacter kombuchae, the bacterial strain Gluconacetobacter xylinus, which is also the most extensively researched and documented in this context, is particularly suitable for the production of nanocellulose gels. The nanocellulose gel is produced by microorganisms at the interface between air and a nutrient medium containing D-glucose. In alternative production processes discussed below, sucrose in aqueous solution is the source of carbon. The bacteria extrude the cellulose in the form of fibrils, which aggregate to form fibers at the interface between the culture medium and air. This creates a three-dimensional intertwined fiber network that consists of about 99% water and 1% nanocellulose.

Due to the special material properties of this biopolymer, an extremely high level of biocompatibility, the structural similarity to the body's own protein-based tissue, the variety of shapes and numerous modification options, it is already used in pharmacy, medicine, cosmetics and food chemistry. Bacterial nanocellulose can be sterilized under the usual conditions, is characterized by a high water content and mechanical stability, while the surface and consistency are described as pleasantly soft and particularly smooth. In cosmetics, for example, it is utilized, enriched with active ingredients and vitamins, in the form of face masks. In medicine, blood vessels, implants and wound dressings made of bacterial nanocellulose are being researched and used.

### REFERENCES

K.-Y. Lee, J. J. Blaker, A. Bismarck: Surface functionalisation of bacterial cellulose as the route to produce green polylactide nanocomposites with improved properties, Composites Science and Technology (2009);
D. Klemm, D. Schumann, F. Kramer, N. Heßler, M. Hornung, H.-P. Schmauder, S. Marsch: Nanocelluloses as Innovative Polymers in Research and Application. Advances in Polymer Science (2006), 205 (Polysaccharides II);
H. Wang, F. Guan, X. Ma, S. Ren: Production and performance determination of modified bacterial cellulose, Shipin Keji (2009), (5), 28-31;
N. Hessler, D. Klemm: Alteration of bacterial nanocellulose structure by in situ modification using polyethylene glycol and carbohydrate additives, Cellulose (Dordrecht, Netherlands) (2009), 16(5), 899-910;
D. Klemm, D. Schumann, F. Kramer, N. Haler, M. Hornung, H.-P. Schmauder, S. Marsch: Nanocelluloses as Innovative Polymers in Research and Application. Advances in Polymer Science (2006), 205 (Polysaccharides II), 49-96;
M. Seifert: Modifizierung der Struktur von Bakteriencellulose durch die Zusammenstellung des Nährmediums bei der Kultivierung von Acetobacter xylinum [EN: Modification of the structure of bacterial cellulose by the composition of the nutrient medium in the culturing of Acetobacter xylinum].

Nanocellulose gels according to the invention in the form of purified nonwoven materials can be used for massage purposes, are used in physiotherapy, complementary medicine, osteopathy, body therapy (also as a cooling or heating pad, massage aid, haptic stimulant) and in personal hygiene (wet wipes, refreshing wipes, probiotic washcloth), for disinfection (loaded with disinfectants). as a stimulation aid or protective film and actively for medical purposes. In addition, a nonwoven material made of nanocellulose gel can be used as a massage glove, washcloth or a means for applying cosmetic products (lotions, creams, oils) to large areas. There is an increased need for nanocellulose gels that can also, but not exclusively, be individually loaded by a user after synthesis of the gels. An unloaded nanocellulose gel is required for this post-modification. The synthesized hydrogel must be as free as possible of bacterial residues that may adhere during synthesis of the gel or be retained in the fibrous network. Other impurities, in particular those that are harmful to health, must also be at least below a limit value. At the same time, during the cleaning procedure, it must be taken into account that the hydrogel is an organic substance, so the cleaning procedure must be appropriately gentle in order to obtain the desired properties.

It is therefore an object of the present invention to provide processes for the production of a nonwoven material consisting of bacterial nanocellulose, in which the hydrogel produced is largely free of foreign matter. At the same time, the process should be quick and easy to carry out, as well as being inexpensive and environmentally friendly.

It is also an object of the present invention to provide a dimensionally stable hydrogel consisting of bacterial nanocellulose that is largely free of foreign matter and can be produced quickly, inexpensively and in an environmentally friendly manner.

The object is achieved by means of the process for the production of a nonwoven material consisting of bacterial nanocellulose according to claim 1. Advantageous embodiments of the invention are set forth in the dependent claims.

The process for the production of a nonwoven material consisting of bacterial nanocellulose has four process steps: In the first process step, a sugar-containing solution is provided. Fructose or sucrose can serve as a carbon source, in the simplest case glucose in an aqueous and acidic buffered nutrient medium, crystalline D-glucose being dissolved in water with, for example, sodium hydrogen phosphate and citric acid at a concentration of 2% by weight to 20% by weight.

This results in a buffered pH value in the slightly acidic range. In the second process step, the sugar-containing solution is inoculated with a strain of bacteria. Nanocellulose gels are preferably synthesized using microbial strains such as Gluconacetobacter, Enterobacter, Agrobacterium, Pseudomonas and Rhizpobium. The strain of bacteria Gluconacetobacter xylinus, also known as Komagataeibacter xylinus, is particularly suitable for the production of nanocellulose gels. Possible strains of bacteria to use are also Gluconacetobacter kombuchae, Komagataeibacter hansenii, Gluconobacter oxydans, Saccharomyces ludwigii, Saccharomyces apiculatus, and Saccharomyces cerevisiae. In the third process step, the solution is cultured, i.e. conditions are created and maintained that ensure the bacteria can metabolize the nutrients. The culturing takes place in the time period of 2 to 25 days. The dimensionally stable hydrogel is produced by microorganisms at the interface between air and the nutrient medium. The bacteria extrude the cellulose in the form of fibrils, which aggregate to form fibers at the interface between the culture medium and air. This creates a three-dimensional intertwined fiber network that consists of about 99% water and 1% nanocellulose. In the fourth process step, the hydrogel resulting from the culturing is washed in order to achieve a degree of purity that is harmless to health. Depending on the production process using different culture media and strains of bacteria, the culturing of bacterial nanocellulose leads to impurities, which are addressed with a purification process step after the culturing. Such a process step is necessary in order to utilize the bacterial nanocellulose as a nonwoven material, raw material, formed object or loadable carrier for use on the skin.

Further components for the optional loading or alternative culturing of the hydrogel include at least one further organic acid at a concentration of 0.1% by weight to 5% by weight selected from the group consisting of: gluconic acid, glucuronic acid, dextrorotatory (L+) lactic acid, tartaric acid, folic acid, oxalic acid, usnic acid, succinic, malic, malonic and citric acid, as well as additionally a trace element (e.g. potassium, calcium, copper, zinc, manganese, cobalt) at a concentration of 1 ppm to 100 ppm. In addition, the solution can contain at least one vitamin (e.g. vitamin B1, vitamin B2, vitamin B3, vitamin B6, vitamin B12, vitamin C, vitamin D, vitamin E, vitamin K).

According to the invention, the hydrogel produced by the culturing is washed in an alkali with a 5% by weight to 50% by weight alkali for a period of time of 5 min to 400 min at a temperature of 37° C. to 142° C. The temperature interval for the cleaning procedure is preferably 90° C. to 142° C., particularly preferably 100° C. to 142° C. The duration of the washing procedure is preferably 60 min to 400 min, particularly preferably 120 min to 400 min. The hydrogels produced by this process according to the invention can be washed off very easily due to their surface structure and, particularly in their synthesized pure form, are insensitive to cleaning agents which are also used on the skin (soap, dish washing detergent, etc.). The hydrogels can be boiled in water or sterilized with hot steam and cleaned in the dishwasher without deformation. They are therefore reusable and the hydrogels are completely biodegradable if disposed of properly.

In a refinement of the invention, the alkali is a 5% by weight to 50% by weight caustic soda solution. Caustic soda solution, i.e. a solution of NaOH (sodium hydroxide) in water, is a standard material in the chemical industry and is therefore available and inexpensive. NaOH is solid in its pure state and is therefore easy to transport. Disposal is also easily possible by neutralizing with acids or sufficiently strong dilution. Washing with caustic soda solution at a concentration and duration tailored to the strength and nature of the hydrogel causes the cell structure of the hydrogel to be more flexible, softer and smoother, without the stability and water retention capacity being significantly reduced for its purpose.

In a further development of the invention, a relative movement between the washing solution and the hydrogel is generated during the washing procedure. Due to the relative movement, impurities adhering to the hydrogel and embedded in the hydrogel are removed more quickly and thoroughly than with a static cleaning procedure.

In a further embodiment of the invention, the washing procedure is carried out in two steps. The two steps of the washing procedure differ in particular with regard to the concentration of the washing solution and the temperature of the washing solution and the duration of the washing procedure, or at least in one of the parameters mentioned. In the first step (preliminary cleaning), a 40% by weight to 50% by weight caustic soda solution is used at a temperature at or up to 15° C. below the boiling point of the caustic soda solution used. The boiling point of a 45% by weight caustic soda solution is 142° C. The duration of the first step of the cleaning procedure is between 1 min and 150 min, preferably between 100 and 140 min. In the second step, a washing solution with a lower concentration, lower temperature and longer duration is used. The concentration of the caustic soda solution is in the range from 0.4% by weight to 8% by weight, the temperature is between 37° C. and 100° C. and thus below the boiling point of the washing solution used. The duration is 1 h to 400 min.

In a refinement of the invention, the washing solution is replaced between the first step and the second step of the washing procedure. If the washing procedure is carried out with the same washing solution, it makes sense to replace the solution in order to reduce the concentration of the unwanted foreign matter.

In an advantageous embodiment of the invention, following the washing procedure, optionally multiple rinsing with distilled water for 30-120 minutes at 80° C. and sterilization (of the hydrogel) are carried out. The cleaning procedure can optionally be completed with a sterilization to kill other microorganisms and to ensure the longest possible shelf life in the packaging. Sterilization can be done in an autoclave.

In a refinement of the invention, the sterilization is carried out using hot steam. The cleaning procedure can optionally be completed with a sterilization, for example with hot steam for 20 minutes at 121° C., in order to kill other microorganisms and to ensure the longest possible shelf life in the packaging. The sterilization can take place at least partially in an autoclave.

The object is further achieved by the nonwoven material made of a hydrogel according to claim 8.

The nonwoven material according to the invention made of a bacterial nanocellulose has a water content of between 80% by weight and 99.5% by weight and a cellulose content of between 0.5% by weight and 20% by weight. According to the invention, the nonwoven material has a foreign matter content of between 0.1% by weight and 15% by weight. In the context of this document, said foreign matter is unusual components that are intentionally or unintentionally components of the nonwoven material.

In a refinement of the invention, the foreign matter contains impurities and loads. Impurities are unintentional components and are largely removed from the nonwoven material during the washing procedure. Loads, on the other hand, are intentional components and are applied to and introduced into the nonwoven material during or after the synthesis procedure of the nonwoven material. Such nonwoven materials are found in cosmetics, for example, enriched with active ingredients and vitamins in the form of skin pads and active ingredient carriers.

Depending on the strain of bacteria, nutrient medium, culturing temperature and duration as well as other parameters, the nonwoven material also has various proportions of the corresponding nutrient solution, which may include various acids and chemical additives, yeast, vitamins or organic residues (for example from plants such as tea, blossoms, fruits or coconut). In certain cases, such as an active, acidic-probiotic load of the nonwoven material (see kombucha culture) or active plant components (comparable to CBD from cannabis in-situ instead of in post-modification), this can be desirable.

In a further embodiment of the invention, the impurities have a content of 0.05% by weight to 1% by weight. In a further configuration of the invention, the impurities have a content of 0.1% by weight to 0.5% by weight. In a further embodiment of the invention, the impurities contain one or more substances from the group of acids mentioned above, trace elements, yeasts, vitamins, and organic or inorganic coloring particles, probiotics, antifungals, disinfectants, alcohol, aloe vera, hyaluronic acid, essential oils, extracts from leaves, roots and fruits and skin particles or body fluids (after use on the skin).

In a further embodiment of the invention, the impurities contain biological impurities and/or chemical impurities. The use of gram-negative strains of bacteria during the synthesis of the nonwoven material carries the risk of endotoxin contamination of the end products, i.e. decomposition products from the outer cell membrane of bacteria that can trigger undesirable reactions in the human organism. These are present in the nonwoven material according to the invention only in a harmless concentration.

The production process also optionally includes the following steps:

Dissolving, in a flat-bottomed vessel, crystalline glucose at a concentration of 2% by weight to 20% by weight, sodium hydrogen phosphate and citric acid in water to form a buffered pH of between pH 4 to pH 7, introducing a dry mixture of peptone and a yeast extract each having a concentration of between 0.1% by weight and 5% by weight into the buffered aqueous solution, stirring the solution until the peptone has completely dissolved and the yeast extract is completely suspended. Sterilization by autoclaving at 121° C. for 20 minutes. Inoculating with the strain of bacteria Gluconacetobacter xylinus, culturing the solution between 2 days and 25 days until a hydrogel forms at the interface of nutrient medium and air, decanting the aqueous solution, washing the hydrogel, purification and then optionally placing the hydrogel in an aqueous solution containing dyes, flavorings, fragrances and active ingredients for between 30 minutes and 30 days, and finally sterilization with hot steam.

In an alternative process, instead of introducing glucose, peptone, yeast, sodium hydrogen phosphate and citric acid, the following step is performed:

Introducing a powder containing 2% by weight to 10% by weight of an extract from black tea, green tea and/or cannabis tea, 90% by weight-98% by weight sucrose and/or glucose, 1% by weight to 5% by weight fruit or vegetable powder, dried leaves and blossoms and dried herbal flavorings and active ingredients.

Inoculation is carried out by adding dried microbes or a liquid solution containing them, at least one species selected from the group consisting of: Gluconacetobacter xylinus, Gluconacetobacter kombuchae, Komagataeibacter hansenii, Gluconobacter oxydans, Saccharomyces ludwigii, Saccharomyces apiculatus or Saccharomyces cerevisiae. In addition, at least one other organic acid is added at a concentration of 0.1% by weight to 5% by weight, selected from the group consisting of: gluconic acid, glucuronic acid, dextrorotatory (L+) lactic acid, tartaric acid, folic acid, oxalic acid, usnic acid, succinic, malic, malonic and citric acid. The sum of the proportions by weight of the components is 100% by weight.

Alternatively, instead of introducing glucose, peptone, yeast, sodium hydrogen phosphate and citric acid, the following step can be carried out: Introducing a solution of 300 g of white refined beet sugar or cane sugar to 21 of coconut water, and 120 ml of concentrated anhydrous acetic acid.

In a further configuration of the process, instead of introducing glucose, peptone, yeast, sodium hydrogen phosphate and citric acid, and instead of inoculating with Komagataeibacter xylinus, the following steps are carried out:

Introducing a solution of 5 g of dried cannabis blossoms or leaves boiled in 1000 ml of water with the addition of one teaspoon of coconut oil for 60 min, adding 100 g of sugar (white refined beet sugar or cane sugar), cooling to room temperature and introducing 250 ml of acidic kombucha tea (pH 2.2-pH 3.5) containing an active kombucha culture (such as live Gluconacetobacter kombuchae).

A set for using one of the aforementioned production processes comprises 2% by weight to 10% by weight of an extract from black tea, green tea and/or cannabis, 90% by weight-98% by weight sucrose and/or glucose, 1% by weight to 5% by weight fruit or vegetable powder, dried leaves and blossoms, dried herbal flavorings and active ingredients, and dried microbes of at least one type selected from the group consisting of:

Gluconacetobacter xylinus, Gluconacetobacter kombuchae, Komagataeibacter hansenii, Gluconobacter oxydans, Saccharomyces ludwigii, Saccharomyces apiculatus or Saccharomyces cerevisiae.

The set also has at least one other organic acid at a concentration of 0.1% by weight to 5% by weight selected from the group consisting of: acetic acid, gluconic acid, glucuronic acid, dextrorotatory (L+) lactic acid, tartaric acid, folic acid, oxalic acid, usnic, succinic, malic, malonic and citric acid. In this case, the sum of the proportions by weight of the components together is 100% by weight. The set is composed in such a way that a pH value of 3.5 to 7 develops in an aqueous solution.

The process for the production of bacterial nanocellulose using a dry instant mix or the two-component solution described below is novel. Crucial production parameters are standardized herein, which leads to a greatly simplified and plannable result. This process can also be used in the food sector (such as kombucha drinks) or textiles (manufacture of vegan leather or fabrics based on bacterial nanocellulose), since the process step of brewing and cooling the tea is eliminated and the mixing ratio between the ingredients remains constant. The instant mix represents a major simplification, especially for home users.

With its traditional domestic brewing and fermentation culture, kombucha often appears as an undefined culture, but usually includes a desirable probiotic composition of bacterial and yeast strains. However, this can vary greatly due to the nature of wild fermentation. Known ingredients here are:

Gluconoacetobacter xylinus, Gluconoacetobacter kombuchae, Gluconoacetobacter hansenii, Acetobacter xylinoides, Gluconobacter oxydans, Saccharomyces ludwigii, Saccharomyces apiculatus, Saccharomyces cerevisiae.

As organic acids, mention should be made of acetic acid, gluconic acid, glucuronic acid, dextrorotatory (L+) lactic acid, tartaric acid, folic acid, oxalic acid, usnic acid, traces of succinic, malic, malonic and citric acid. Trace elements and minerals include iron, magnesium, sodium, potassium, calcium, copper, zinc, manganese, cobalt and other minerals.

The list of vitamins includes vitamin B1, vitamin B2, vitamin B3, vitamin B6, vitamin B12, vitamin C, vitamin D, vitamin E, vitamin K. Also contained are various amino acids, enzymes, tannins, the enzymes invertase, amylase, catalase, saccharase, rennet and a proteolytic enzyme, antibiotic substances, alcohol and carbonic acid.

The bacterial cultures of kombucha have the special property of being able to assert themselves against foreign bacteria that threaten the system in sufficiently acidic liquid. If they are provided with nutrients, natural dyes, active ingredients and aromas, the growing bacterial cellulose takes on additional properties with its high water absorption (approx. 99% water, 1% cellulose) and its water retention capacity.

Active nanocellulose gels contain living probiotic strains of bacteria; in passive nanocellulose gels the strains of bacteria were killed and removed by purification process steps, and the hydrogels were sterilized by autoclaving (121° C. steam for 15-20 minutes) or electron beam processes.

The combination of 250 ml of acidic kombucha tea [“Fairment Kombucha—Original” pH 2.5-2.8] or a defined nanocellulose gel strain (pH 2.2-3.5) with a suitable active bacterial culture and 25 g of instant powder is suitable for the production of one or more kombucha-based nanocellulose gels with a total mass of 50 g or more in 2-25 days of standing culturing under hygienic conditions and with oxygen supply. The material properties of the kombucha-based nanocellulose gels are similar to the synthesized biopolymers mentioned above.

The liquid by-product is an acidic tea solution with the usual kombucha composition of bacterial and yeast cultures (pH 2.3-4) with proportions of organic aromas, dyes, flavorings and active ingredients (from fruit or vegetables, teas, herbal aromas and active ingredients according to the composition of the instant powder). This solution is now suitable for inoculating and staining, as well as for storing, renovating and maintaining a nanocellulose gel. It can be used to revitalize a sterilized, passive nonwoven material with the probiotic culture.

Due to the high water absorption and the ability to release water under mechanical impact, weight specifications are subject to high fluctuations and can only be used as a guide herein. Growth is also crucially impacted by the container. The shape, the surface, the filling level and the material are factors that determine the properties. Porcelain, plastic, and glass containers are best for growing nanocellulose gels at home.

In addition to synthesized, passive nanocellulose nonwoven material and the probiotically active hydrogels, there is a third process of producing a coconut-based nanocellulose gel, which in turn has similar material properties to the nanocellulose gels mentioned above and can also be loaded in situ or subsequently. Here, too, the production process of fermentation in standing culturing over 2 days to 25 days is suitable, with the nutrient medium (pH 2.3-3.5) being composed as follows: 120 ml of concentrated, anhydrous acetic acid (glacial acetic acid), 300 g of sugar [Naturata Bio-Beet Sugar] (white, refined, or raw cane sugar), 300 ml of Nata Starter (strain of bacteria Gluconacetobacter xylinus), alternatively a kombucha starter or unpasteurized kombucha drink [“Fairment Kombucha—Original”] can be used, also 2 l of coconut water [“Coco Juice Pure Organic”].

A fourth variant of producing bacterial nanocellulose is the use of medicinal cannabis. For this purpose, the nutrient solution is prepared by boiling 5 g of cannabis leaves or blossoms in 1 l of water and a teaspoon of coconut oil for 60 minutes and adding 100 g of sugar [Naturata Organic Beet Sugar] (white, refined, or raw cane sugar). The addition of 250 ml of acidic kombucha tea [Fairment Kombucha—Original pH 2.5-2.8] or a defined nanocellulose gel strain (pH 2.2-3.5) starts the production of nanocellulose, which contains effective cannabinoids in addition to the properties mentioned above. There are many receptors for cannabinoids in the skin and mucous membranes. The medicinally active component cannabidiol (CBD), for example, promotes blood circulation in the tissue, which can lead to greater sensitivity.

The nanocellulose gels, which are produced in different ways, are preferably used in the following product variants: At a size of 150-300 mm×150-300 mm and a thickness of 0.1-3 mm, the wet wipe or protective film has a drained net weight of 12-180 g. At a size of 120-180 mm×120-180 mm and a thickness of 3-10 mm, the washcloth (or massage sponge or haptic stimulant) has a drained weight of 60-180 g.

Both formats (with rounded corners or grown or cut into rectangles) can be combined with a glass or plastic cylinder, which can be filled with warm water or skin care products. A washing glove results from the combination of two cloths (such as square, rectangular, in the shape of a hand, etc.), which can be cut, sewn and folded or pressed in a similar way to textiles.

As a material unit for individual further processing, the nonwoven material has a length of 150-400 mm and a width of 120-300 mm when not rolled. In rolled form, a cylindrical shape of the specified length and, depending on the thickness of the material (0.5-25 mm), an outside diameter of 30-120 mm, with a drained net weight of 100-1200 g is obtained. Size specifications vary, since the products are both industrially assembled and can also be individually adapted to the needs of consumers. By culturing in an appropriately shaped vessel—preferably made of glass or a food-safe plastic—foldable and rollable flaps (such as circular, oval, rectangular, square, diamond-shaped, triangular) can be produced that vary in thickness, depending on the duration of the fermentation, the temperature and the addition of nutrients. With the appropriate devices and vessels, various components such as cylinders, hose-like covers and protectors can also be designed using the process described.

From a solidly grown block or a thin membrane, shapes can be modeled with the help of cutting and milling tools (knives, scissors, die cutters, lasers, hole punches) or 3D printing processes that would not result from purely organic growth. This results in a large number of other components in a modular system.

These nanocellulose gel components can be connected to one another using rubber bands, cords, cuffs, rings, clamps, staples or the technique of sewing. Massage tools, bags, vessels such as glasses, bottles or tubes can be combined to give shape and stability or to be used for storage.

Tools such as templates made of plastic, glass or cork can also be used in culturing to shape the bacterial nanocellulose gel growing on the surface while it is still growing. This can represent the final shape or individual components for a specific design of more complex nanocellulose gel-based models. With lasers, stamping irons, embossing tools and branding irons, objects made of bacterial nanocellulose can be provided or designed with model numbers, production dates or other information.

The reduction of plastic waste and use of non-renewable raw materials is an ecological opportunity for the technology to grow when used on the skin. The possibility of resource-saving individual domestic production, as well as the possibility of choosing from different production processes in order to be able to access regionally produced and easily available raw materials on an industrial scale, means that packaging, delivery routes and thus emissions can be avoided.

The ideal duration of the purification that follows culturing and the concentration of NaOH (in distilled water) depends on the exact composition of nutrients and bacteria used, whether they are composites, hybrids or pure cultures.

In one embodiment of the process according to the invention, a single-step purification process is used. For each millimeter of thickness of the nonwoven material, a 2-hour exposure time at 85° C. in 100 ml of 0.8% by weight NaOH solution per cubic centimeter of cellulose achieves a reliable termination of all bacterial activity in all of the processes mentioned. Depending on the desired degree of purity, this process step can be repeated with replacement of the sodium hydroxide solution—or take place in dynamic flow—until this NaOH solution absorbs no or only small amounts of detectable impurities from the cellulose produced. The nonwoven material is then rinsed with distilled water and, depending on the intended use, the pH is adjusted to the desired value between pH 4 and pH 7—preferably to a skin-neutral value of around 7, so that the pH can be easily readjusted later by appropriate loading. Optionally, citric acid can also be used for this neutralization step in addition to the distilled water.

The nonwoven material cleaned in this way has an (intended) loading of 8% by weight and 1.5% by weight of impurities. The purification can optionally be completed with a sterilization, for example with hot steam for 20 minutes at 121° C., in order to kill other microorganisms and to ensure the longest possible shelf life in the packaging.

In terms of the best possible shelf life for retailers, the packaging should ideally be vacuum-packed in water-impermeable film, without air supply in liquid (distilled water or in loading solution) and, in terms of sustainability, in a reusable, lockable cylindrical container, or the bacterial nanocellulose freeze-dried in water-repellent or waterproof packaging for later swelling.

In a further embodiment of a one-step cleaning process, the nonwoven material is cleaned at 110° C. for 240 minutes with a 45% by weight NaOH solution. The amount of NaOH solution is also 100 ml per cubic centimeter of cellulose. After cleaning, the nonwoven material is also rinsed with distilled water and optionally citric acid. The nonwoven material cleaned in this way has a loading of 9.5% by weight and 0.8% by weight of impurities.

In a further embodiment of the process according to the invention, a two-step cleaning process is used. In the first step (preliminary cleaning), an NaOH solution containing 50% by weight NaOH is used, the duration of action is 135 min and the temperature is 127° C. In the second cleaning step, the nonwoven material is cleaned with an 8% by weight NaOH solution at a temperature of 85° C. for 240 min. The nonwoven material cleaned in this way has a loading of 10.5% by weight and 0.27% by weight of impurities.

1. A process for the production of a hydrogel consisting of bacterial nanocellulose, comprising the following steps:
providing a sugar-containing solution
inoculating said sugar-containing solution with a strain of bacteria
culturing said solution
washing the hydrogel resulting from the culturing,
characterized in that
the hydrogel produced by the culturing is washed in an alkali with a content of 5% by weight to 50% by weight at 37° C. to 142° C. for 5 min to 400 min.

2. The process for the production of a film consisting of bacterial nanocellulose according to claim 1,
characterized in that
the alkali is a 5% by weight to 50% by weight caustic soda.

3. The process for the production of a film consisting of bacterial nanocellulose according to claim 1,
characterized in that
a relative movement between the washing solution and the hydrogel is generated during the washing procedure.

4. The process for the production of a film consisting of bacterial nanocellulose according to claim 1,
characterized in that
the washing procedure is carried out in two steps.

5. The process for the production of a film consisting of bacterial nanocellulose according to claim 4
characterized in that
the washing solution is replaced between the first and the second stages of the washing procedure.

6. The process for the production of a film consisting of bacterial nanocellulose according to claim 1,
characterized in that
sterilization is carried out after the washing procedure.

7. The process for the production of a film consisting of bacterial nanocellulose according to claim 6,
characterized in that
sterilization is carried out with steam.

8. A nonwoven material made of a hydrogel with a water content of between 80% by weight and 99.5% by weight, and a cellulose content of between 0.5% by weight and 20% by weight,
characterized in that
the nonwoven material has a foreign matter content of between 0.01% by weight and 15% by weight.

9. The nonwoven material made of a hydrogel according to claim 8,
characterized in that
said foreign matter contains impurities and loads.

10. The nonwoven material made of a hydrogel according to claim 9,
characterized in that
the impurities have a content of 0.01% by weight to 2% by weight.

11. The nonwoven material made of a hydrogel according to claim 9,
characterized in that
the impurities have a content of 0.05% by weight to 1% by weight.

12. The nonwoven material made of a hydrogel according to claim 9
characterized in that
the impurities have a content of 0.1% by weight to 0.5% by weight.

13. The nonwoven material made of a hydrogel according to claim 9,
characterized in that the impurities (or load) have one or more substances from the group of acids (acetic acid, gluconic acid, glucuronic acid, dextrorotatory (L+) lactic acid, tartaric acid, folic acid, oxalic acid, usnic acid, succinic, malic, malonic and citric acid), trace elements and minerals (iron, magnesium, sodium, potassium, calcium, copper, zinc, manganese, cobalt), yeast extract and vitamins (vitamin B1, vitamin B2, vitamin B3, vitamin B6, vitamin B12, vitamin C, vitamin D, vitamin E, vitamin K, vitamins), peptone, sodium hydrogen phosphate, carbonic acid, as well as organic or inorganic coloring particles, probiotics, antifungals, disinfectants, alcohol, aloe vera, hyaluronic acid, essential oils and fragrances, extracts from leaves, roots and fruits as well as skin particles or body fluids (after use on the skin).

14. The nonwoven material made of a hydrogel according to claim 9,
characterized in that
the impurities contain biological impurities and/or chemical impurities.
